(19) United States
(12) Patent Application Publication
(10) Pub. No.: US 2022/0047611 A1
(43) Pub. Date: Feb. 17, 2022

(54) Capsule, Tablet or Pill

(71) Applicant: Parapharm Development Limited, Windsor Berkshire (GB)

(72) Inventors: Susan A.S. Lloyd, Faringdon, Oxfordshire (GB); John Whitfield, Clacton on Sea Essex (GB)

(21) Appl. No.: 17/275,665

(22) PCT Filed: Sep. 11, 2018
(86) PCT No.: PCT/EP2018/074476

§ 371 Date: Mar. 11, 2021

(51) Int. Cl. A61K 31/593; A61K 31/07; A61K 31/355; A61K 31/122; A61K 47/14

(52) U.S. Cl. CPC A61K 31/593; A61K 31/07; A61K 47/14; A61K 31/122; A61K 31/355

(57) ABSTRACT

Disclosed is a capsule, tablet or pill comprising a formulation at least two of the fat-soluble vitamins vitamin A, vitamin D, vitamin E and vitamin K; and one or more kinds of medium-chain triglyceride; also disclosed are foods, drinks and pharmaceutical compositions comprising the capsule, tablet or pill; and methods of treatment of a patient comprising administration of an effective amount of the capsule, tablet or pill. The capsule, tablet or pill is particularly, but not exclusively, suitable for use in treating cystic fibrosis patients.

### FIELD OF THE INVENTION

The present invention relates to a capsule, tablet or pill comprising a formulation which is particularly, although not exclusively, designed to treat patients having cystic fibrosis. The invention also relates to the use of the capsule, tablet or pill to treat patients having cystic fibrosis, and to a method of treating patients having cystic fibrosis using the capsule, tablet or pill.

### BACKGROUND

Cystic fibrosis (CF) is a genetically inherited condition as a result of mutation in the CTFR gene. The CTRF gene provides instructions that controls the movement of salt and water in and out of cells resulting in build-up of thick sticky mucus in lungs, GI and other organs. When fats are taken into the stomach they tend to float on the aqueous stomach contents. This is responsible for the well-known effect of gastric discomfort and regurgitation and fishy taste that occurs after taking standard fat capsules. This is a lasting effect and delays the passage of fatty material out of the stomach.

Sufferers of CF have a problem that fat-soluble vitamins (FSVs) are not efficiently absorbed, especially those who are also pancreatic insufficient. Low vitamin levels have their own associated clinical conditions. FSVs include vitamins A, D, E and K.

Therefore, CF patients often require vitamin supplementation and are commonly advised to maintain a normal to high fat diet to assist the absorption of FSVs to avoid deficiencies of these vitamins.

There is a general aim in the art to supplement CF patients with sufficient vitamin A, D, E and K to provide the patient with plasma levels of these vitamins at the upper limit of the normal range (see Nutritional Management of Cystic Fibrosis, 2^{nd} Edition—September 2016 from the Cystic Fibrosis Trust). These guidelines recommend that adults take starting daily vitamin doses of:

1500-10,000 IU vitamin A;
400-5,000 IU vitamin D3;
150-500 IU vitamin E; and
5-10 mg vitamin K1.

A variety of vitamin supplements are available. However, the efficacy of these supplements varies. A number of clinical assessments may be made initially in order to assess the levels of supplementation tablets or capsules or other forms needed to achieve sufficient uptake of the FSVs. Adjustments can only be made following such assessment. Subsequently, monitoring of vitamin levels is routine. This is onerous for CF patients.

Furthermore, presently known supplements have an additional “pill burden” which leads to excess ingestion of unnecessary or even unwanted excipients. The need to administer 15-20 tablets or pills or capsules per day also exacerbates problems with patient compliance.

It is desirable to provide a supplement of FSVs that provides an improved absorption of the vitamins by CF patients. It is further desirable to provide a supplement wherein the absorption of the necessary vitamins by the CF patients is more predictable than the absorption levels presently provided by known supplements. It is also desirable to reduce the burden on the patient.

The present invention has been devised in light of the above considerations.

### SUMMARY OF THE INVENTION

Fats and fat soluble vitamins are absorbed in the proximal part of the small bowel, the duodenum and jejunum, where the intra-luminal environment changes from the acid pH of the stomach to an alkaline pH. This allows the rapid exposure of the (fat soluble vitamin) contents of an appropriately designed formulation, to the surrounding high concentration of bile acids and pancreatic enzymes. These act together to form small fat vitamin-containing micelles which can then be absorbed by the enterocytes that line the gut wall in the duodenum and jejunum.

The inventors have studied this process and have found that using medium chain triglycerides (MCTs) as a diluent with FSVs A, D, E and/or K aids the optimal absorption of the vitamin contents and avoids gastric discomfort and regurgitation in CF patients. Advantageously, a lack of excipients in preferred formulations also removes any bitter or metallic taste in the mouth of CF patients.

The present invention has been devised based on these findings.

Accordingly, in a first aspect the present invention concerns a capsule, tablet or pill comprising a formulation comprising at least two of the fat-soluble vitamins vitamin A, vitamin D, vitamin E and vitamin K; and one or more kinds of medium-chain triglyceride. Substantially preferably, at least two fat-soluble vitamins are dissolved in the one or more kinds of medium-chain triglyceride. Particularly suitably, the formulation comprises each of the fat-soluble vitamins vitamin A, vitamin D, vitamin E and vitamin K.

In a second aspect, the invention concerns a food or drink comprising the capsule, tablet or pill of the first aspect.

In further aspects, the invention concerns a capsule, tablet or pill of the first aspect, or the food or drink of the second aspect, for use in therapy; and a pharmaceutical composition comprising the formulation of the first aspect or the food or drink of the second aspect. In particularly preferred embodiments, these further aspects are suitable for use in the treatment of cystic fibrosis patients. Accordingly, still further aspects relate to methods of treatment of patients comprising administration of the capsule, tablet or pill of the first aspect, or the food or drink of the second aspect, to a patient in need thereof.

In this specification, “capsule” is used, although it should be understood that the term encompasses “tablet” and “pill” unless expressly provided otherwise. That is, where features are described in relation to a capsule, they are generally also applicable to a tablet or pill of the invention unless context dictates otherwise.

The invention includes the combination of the aspects and preferred features described except where such a combination is clearly impermissible or expressly avoided.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

The present inventors believe that certain preferred embodiments described herein are capable of providing increased absorption of FSVs, especially for CF patients. Particularly through the use of MCTs, the location of delivery of the FSVs may be biased in the lower gastrointestinal tract, instead of in the stomach, and may achieve a generally increased absorption. They further believe that some of the embodiments described herein may have other benefits, including minimising undesirable taste and odours, avoiding reflux from the stomach, low risk of side-effects, and improved quality of life arising from improved absorption of FSVs. Further, they hope that regular administration of the capsules described herein may mean that a CF patient could eventually have to undergo less rigorous monitoring in order to check their FSV levels, because the absorption will be more predictable.

### Formulation

### Fat-Soluble Vitamins

A capsule of the present invention comprises a formulation that contain a combination of FSVs. Suitably, the formulation comprises two or more of vitamin A, vitamin D, vitamin E and vitamin K, and preferably three and most preferably each of these. Most preferably, the formulation comprises vitamin A (retinyl palmitate), vitamin D3 (cholecalciferol), vitamin E (DL-alpha tocopheryl acetate) and vitamin K1 (phytomenadione).

These vitamins and their structures are known to the skilled person. We include some non-limiting explanations of each. Reference may also be made to the Encyclopedia of Dietary Supplements, 2^{nd} Edition, Coates et al. (2010), pages 778-860.

Vitamin A is the name given to retinol-based compounds, and as used herein can include one or more of retinol, retinal, retinoic acid, beta-carotene and derivatives or precursors thereof. Vitamin A has a number of functions in the body, including e.g. for vision and growth.

Retinol (all trans) has the structure shown in Formula 1 below, where R indicates various substituents at carbon 15 giving rise to retinol, retinyl esters, retinal, and retinoic acid. Some of these molecules also exist naturally in several isomeric forms such as 9-cis, 11-cis, and 13-cis-isomers. Such isomers are intended to be encompassed unless context dictates otherwise.

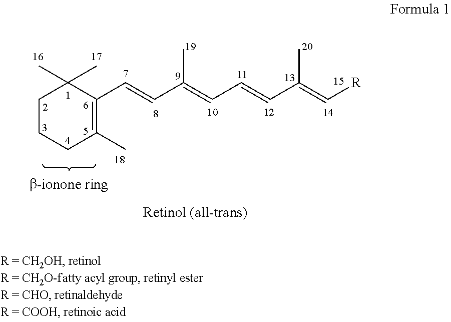

Typically, the present invention includes one or more compound derivatives or precursors of vitamin A, and suitably a derivative or precursor that releases retinol in the body following absorption. The skilled person will be aware of such vitamin A derivatives or precursors in the art. The retinoid derivatives or precursors are defined above in Formula 1. Particularly suitable derivatives or precursors include esters. Suitably, the ester can be an ester of retinol and an acid such as palmitic acid. Most suitably, the formulation used in the present invention includes retinyl palmitate. The retinyl ester can preferably be a synthetic ester, such as retinyl acetate. Retinyl esters tend to be stable, and less oxygen sensitive than unesterified retinols. Therefore, use of a retinyl ester helps to increase the shelf life of the formulation and therefore the capsule, tablet or pill.

Humans can also ingest vitamin A in a form often referred to as provitamin A. Biochemically, this consists of several specific types of carotene, of which 3-carotene is the major form in most diets and thus warrants special mention here. Provitamin A is also a precursor or derivative of vitamin A, though it is less preferred in the present invention.

Vitamin D is the name given to fat-soluble secosteriod compounds. As used herein, it can include one or more of vitamins D₁ to D₅, particularly vitamin D₂ (ergocalciferol) and vitamin D₃ (cholecalciferol). Vitamin D has a number of functions in the body including assisting in absorption of e.g. calcium. Vitamin D₂ has the structure shown in Formula 2, and vitamin D₃ has the structure shown in Formula 3:

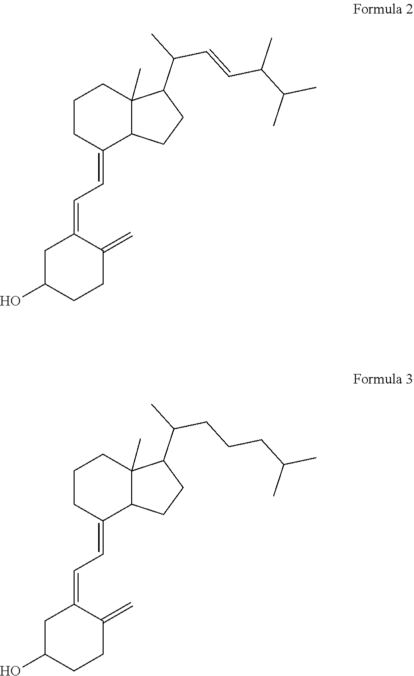 [SMILES: C=C1CCC(O)C/C1=C/C=C1\CCCC2(C)C1CCC2C(C)/C=C/C(C)C(C)C ; C=C1CCC(O)C/C1=C/C=C1\CCCC2(C)C1CCC2C(C)CCCC(C)C]

Preferably, the capsule, tablet or pill according to the invention contains vitamin D₃.

Vitamin E as used herein can include tocopherols and tocotrienols, derivatives and precursors thereof. Vitamin E has a number of functions in the body e.g. as an antioxidant in the body. The most biologically active form of vitamin E is α-tocopherol, which has the structure shown in Formula 4:

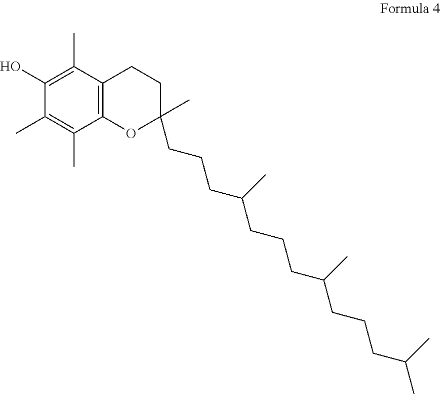 [SMILES: Cc1c(C)c2c(c(C)c1O)CCC(C)(CCCC(C)CCCC(C)CCCC(C)C)O2]

Typically, the present invention includes one or more compound derivatives or precursors of vitamin E, and suitably y tocopherol or a tocopherol or derivatives or precursors thereof. The skilled person will be aware of such vitamin derivatives or precursors in the art. Suitable derivatives or precursors include acetates. Most suitably, the capsule of the present invention includes DL-alpha tocopheryl acetate and DL-alpha tocopherol.

Vitamin K has a number of functions in the body and includes e.g. affecting the ability of the body to govern blood coagulation. Vitamins K₁ and K₂ are particularly suitable for use in the present invention, especially vitamin K₁ which is also known as phylloquinone and has the structure shown in Formula 5:

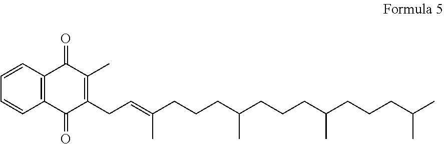 [SMILES: CC1=C(C/C=C(\C)CCCC(C)CCCC(C)CCCC(C)C)C(=O)c2ccccc2C1=O]

Particularly preferred is a capsule including DL-alpha tocopheryl and the acetate derivative thereof, retinyl pamitate, phylloquinone and cholecalciferol.

### Amounts

In general, it is preferred for the vitamins of the present formulation to be present in therapeutically effective amounts. As used herein, mcg is microgram (or μg). The amounts are per 1 capsule, tablet or pill - abbreviated here as per 1 capsule as described above.

Suitably, vitamin A (e.g. retinyl palmitate) can be present in an amount of between 500 and 2500 mcg per capsule, such as between 750 and 2000 mcg, or 1000 and 1750 mcg per capsule. For example, the vitamin A can be present in an amount of up to 2500, such as 2250, 2150 or 2000 mcg per capsule. The vitamin A can be present in an amount of at least 500, such as 750 or 850 or 1000 mcg per capsule.

Suitably, vitamin D (e.g. vitamin D3) can be present in an amount of between 10 and 75 mcg per capsule, such as between 15 and 50 mcg or 25 and 40 mcg per capsule. For example, the vitamin D can be present in an amount up to 75 mcg, such as 70, 65 or 60 mcg per capsule. The vitamin D can be present in an amount of at least 10, such as 15, 20 or 25 mcg per capsule.

Suitably, vitamin E (e.g. DL-alpha tocopheryl acetate) can be present in an amount of between 20 and 200 mg per capsule, such as between 30 and 175 or between 50 and 150 mg per capsule. For example, the vitamin E can be present in an amount of up to 200, such as 175 or 150 mg per capsule. The vitamin E can be present in an amount of at least 20, such as 40, 60 or 80 mg per capsule.

Suitably, vitamin K (e.g. phytomenadione) can be present in an amount of between 1 and 10 mg per capsule, such as between 2 and 8 mg per capsule or between 4 and 6 mg per capsule. For example, vitamin K can be present in an amount of up to 10, such as up to 9, 8 or 7 mg per capsule. The vitamin K can be present in an amount of at least 1, such as 2, 3 or 4 mg per capsule.

As the skilled person will appreciate, the amounts of active vitamins can alternatively be written in term of the International Unit (IU). The precise conversion varies according to the kind of vitamin. For example, 1 mcg is equivalent to about 3.3 IU for retinyl palmitate; to about 40 IU for vitamin D3; and to about 1.5 IU for DL-alpha tocopheryl acetate. The conversions are known to persons skilled in the art.

MCTs are used in the present invention as a diluent for the FSVs. Accordingly, the amounts of MCTs will vary to accommodate the desired concentrations. Typically, the MCTs will be present in an amount of up to about 85%, such as about 70%, and preferably up to about 50% by weight of the formulation. Typically, the MCTs will be present in an amount of at least about 10%, at least about 30% or at least about 40% by weight of the formulation. For example, MCTs may be present in an amount between 25 and 80%, between 35 and 75%, or between about 35 and 55% by weight of the formulation. Illustratively, MCTs may be present in an amount of between 20 and 300 mg per capsule, such as between 30 and 250 or 40 and 200 mg per capsule. For example, MCTs may be present in an amount of up to 400, such as 300 or 200 mg per capsule. The MCTs may be present in an amount of at least 20, such as 25, or 35 mg per capsule.

When each of vitamins A, D, E and K are present, vitamin E may be present in the largest proportion of the formulation. Generally, vitamin E makes up at least 50% of such formulations, and up to 90%, for example between 50 and 80, or between 60 and 70%. Vitamins A and K may be present in substantially equal amounts by wt %, usually at least 0.75% and up to 10%, such as between 1 and 5%, such as between 1.5 and 3% of the formulation. Vitamin D may be present in smaller amounts, such as 1% or less e.g. between 0.01 and 1% of the formulation. In general, MCTs make up the remainder, usually at least 20% and less than 50%, such as between 25-45% of the formulation. These are weight percentages (wt %) of the formulation.

Other components that may optionally be present in the formulation are present in relatively minor amounts. Capsules comprise a shell enclosing a filling, which filling typically includes the formulation comprising the active ingredient(s) and optionally may comprise one or more other components.

Examples of these other components that may be present typically include bees' wax and soy lecithin. Typically, such components make up between around 2 and 7 wt % of the capsule filling, but this can be determined by the skilled person and is not limiting herein.

The shell of capsules, tablets or pills can include typical components. By way of non-limiting example, soft shells can include polymeric ingredients such as gelatin or glycerol, and colorants such as titanium dioxide, sodium copper chlorophyllin and black iron oxide. The skilled person can determine suitable amounts, though typically colorants make up a minority portion (e.g. around 0.5-2 wt %) and the polymeric ingredients a majority proportion (e.g. more than 90%, such as 98-99 wt %) of the shell.

### Medium-Chain Triglycerides

The FSVs of the present invention are dissolved in medium-chain triglycerides (MCTs). Without wishing to be bound by theory, the inventors believe that the use of MCTs is important to encourage sufficient FSV to permeate the pili of the digestive tract of a CF patient. That is, it is believed that the MCTs help absorption and improve the solubility of the vitamins when released from the formulation. In contrast, commonly-used excipients such as lactose used in other formulations do not provide such advantages.

The present inventors believe that the use of MCTs may provide other benefits aside from the improved absorption profile, including better taste profile for the subject.

The person skilled in the art understands the scope of MCTs. Broadly, MCTs have a glycerol backbone connected to three fatty acid chains. As used herein, MCTs are triglycerides wherein at least one of the fatty acids has an aliphatic tail of between 6 and 12 carbon atoms. Preferably, at least 2 and most preferably 3 of the fatty acid chains of the MCTs have an aliphatic tail of between 6 and 12 carbon atoms. The aliphatic tail may be saturated or unsaturated and may be branched or linear.

MCTs are obtainable through known sources, for example oils such as palm kernel oil and coconut oil. MCTs are usually in liquid form at room temperature, and this is preferred in the present formulations. Preferred MCTs are derived from coconut oil and palm kernel oil.

### Coating Compositions

The formulation is typically provided with a shell or coating because it is provided as a capsule, tablet or pill. Such shells can contain suitable components known to the skilled person. The coatings generally make up around 25-35 wt % of the total capsule weight.

Exemplary shells contain components including gelatin, glycerol, titanium dioxide, sodium copper chlorophyllin and black iron oxide. Coatings containing such ingredients are particularly advantageous because it has been found possible to provide them in a tasteless, odourless form and with no artificial colouring which can be desirable for certain patients. They may also be provided in a form that is suitable for Halal/Kosher diets.

Suitably, the gelatin may be present in an amount between 50-75 wt % of the shell.

Suitably, the glycerol may be present in an amount between 25-40 wt % of the shell.

Colorants may make up e.g. 1-2 wt % of the shell as noted above. The total wt % should be 100 wt % as understood in the art.

A suitable dose of formulation per capsule is generally provided as between 0.1 and 1.0 mL. In general, preferred doses per capsule are 0.25 mL formulation, for ease of swallowing.

A capsule may suitably have a total weight of between 500 mg and 1 g, such as between 600 mg and 800 mg. Suitably, the described formulations make up between 50-90% of the total capsule weight (the remainder usually being shell coating composition), and preferably between 65-85%.

### Other Preferences

Advantageously, there is no requirement to include salt in either the coating or the filling.

Preferably, the present capsules can be particularly low energy (e.g. 0.1-10 kcal per dose), low-fat (e.g. 0.01-1 g per dose), and/or low-carbohydrate (0-0.1 g per dose), suitable for health-conscious patients. Similarly, the present capsules preferably contain no sugar, no salt and little to no protein (e.g. 0-0.5 g per dose).

Advantageously, the present capsules can be rendered suitable for a wide variety of patients as there is no need to include various substances recognised as allergens, including for example cereals containing gluten, crustaceans or molluscs, nuts including peanuts, mustard, fish and others.

### Medical Applications

The formulations disclosed herein are particularly designed to supplement or assist in the treatment of patients having conditions that lead to difficulties in absorption of FSVs, and particularly preferably in the treatment of CF patients.

Formulations for use in the invention may be provided as pharmaceutical compositions for clinical use and may comprise a pharmaceutically acceptable carrier, diluent or adjuvant. The composition may suitably be formulated for oral routes of administration

In one embodiment, the composition is a pharmaceutical composition comprising at least one compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g. wetting agents), masking agents, colouring agents, flavouring agents, and sweetening agents.

Suitably, the present formulations include at least one or more stabilizers, one or more colorants, and one or more emulsifiers.

The formulation may further comprise other active agents, for example, other therapeutic or prophylactic agents.

Suitable carriers, diluents, excipients, etc. can be found in standard pharmaceutical texts. See, for example, Handbook fo Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients 2nd edition, 1994.

### Routes of Administration

Capsules according to the present invention may be formulated for administration by a number of routes, including but not limited to, oral.

A capsule may be administered alone or in combination with other treatments, either simultaneously or sequentially dependent upon the condition to be treated.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

The present capsules are particularly suitable for oral administration.

In particular, the formulation is provided as a liquid, in the form of a capsule which comprises the liquid.

In particular, it is believed that the liquid-filled capsules may provide an advantage of avoiding reflux taste in the mouth of a patient to whom the formulation is administered. Advantageously, capsules can be prepared using gelatin or can be made suitable for vegetarians.

The capsule of the present invention is capable of providing a CF patient with additional nutrients i.e. it is nutritionally valuable. Suitably, the capsule of the present invention is, or is a constituent of, a pharmaceutical composition. Suitably, the capsule of the present invention is, or is a constituent of, a nutritional supplement. Suitably, the capsule of the present invention is a constituent of a nutritional food or drink, and may be for example dissolved therein.

### Dosages

The capsules of the present invention would usually be administered with the oversight of a medical practitioner, who can determine suitable dosage regimes according to the needs of the individual patient. Factors that determine a suitable dosage includes, for example, the weight, age and nutritional needs of the patient.

Commonly, the present capsules will be taken by adults or children over the age of 3.

Commonly, the present capsules will be taken orally with a meal to enhance absorption.

It is expected that a patient will typically receive between 1-2 capsules or pills of the invention per day.

### Preparation of Formulation

Another aspect of the present invention pertains to methods of preparing the formulations described herein. The method may include admixing the vitamin components, as defined herein, along with MCTs. The method may be any method known to the skilled person.

In general, typical and non-limiting methods may include providing the FSVs, and blending with one or more MCTs such that the FSVs are dissolved in the MCT(s). Further ingredients may be provided if wanted, and subsequently encapsulated into a pre-prepared coating (shell) by methods known in the art. The capsules may optionally be dried and then packaged.

Another aspect of the present invention pertains to methods of making a capsule of the present invention comprising a pharmaceutical composition, the method comprising admixing at least two of the vitamin components and MCTs, as defined herein, together with one or more other pharmaceutically acceptable ingredients well known to those skilled in the art, e.g. carriers, diluents, excipients, etc., and encapsulating as described above.

Since they are formulated as discrete units (e.g. pills, etc.), each unit contains a predetermined amount (dosage) of the vitamins.

The term “pharmaceutically acceptable” as used herein pertains to compounds, ingredients, materials, compositions, dosage forms, etc., which are, within the scope of sound medical judgment, suitable for use in contact with the tissues of the subject in question (e.g. human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

The pharmaceutically acceptable compositions may be prepared by any methods well known in the art of pharmacy. Such methods include the step of bringing into association the formulation with a carrier which constitutes one or more accessory ingredients. In general, the formulations are prepared by uniformly and intimately bringing into association the active compound with carriers (e.g. liquid carriers, finely divided solid carrier, etc.), and then shaping the product, if necessary.

Tablets may be made by conventional means, e.g. compression or moulding, optionally with one or more accessory ingredients. The tablets may optionally be coated or scored and may be formulated so as to provide slow or controlled release of the active compound therein. Tablets may optionally be provided with a coating, for example, to affect release, for example an enteric coating as described below.

### Enteric Coating

Optionally, the capsule, tablet or pill can be provided with an enteric coating. The coating may be in addition to, instead of, or as a constituent of, the shell described above. Typically, in known formulations, an enteric coating is not used for vitamin-containing formulations because typically a person is able to absorb the vitamins sufficiently well. However, as explained above, CF patients do not typically absorb vitamins sufficiently well. [By “sufficiently well” as used here, we mean in general that the majority of the provided vitamins are absorbed by the digestive tract of a person to whom the formulation is provided. The absorbed vitamins are then available for the body to process.] Without wishing to be bound by theory, it is thought that an enteric coating may be suited for use in a CF patient by at least partially delaying release of the FSVs from the formulation until the formulation reaches a suitable part of the digestive tract of the patient. In particular, it is believed that an enteric coating may encourage release of the FSVs nearer the pancreas. In this way, it is believed that an increased proportion of FSVs will be absorbed compared to other known products. Enteric coatings may also provide protection of the FSVs from oxygen and humidity.

An enteric coating suitable for use in the present invention is a polymer-based coating. That is, an enteric coating suitable for use in the present invention comprises polymers. The main polymers used in enteric coatings currently can be broadly classified into cellulose esters, polymethacrylates, and polyvinyl derivatives. These would be suitable for use in the invention. A non-exhaustive list of exemplary, suitable materials for use in enteric coatings according to the invention includes: cellulose acetate trimellitate; methacrylic acid/ethyl acrylate (e.g. those commercially available in e.g. the Edragit® range including Eudragit® L and Eudragit® S); hydroxy methyl cellulose phthalate; hydroxypropyl methyl cellulose acetate succinate; ethyl cellulose; methylcellulose; carboxymethylcellulose; cellulose acetate phthalate; cellulose acetate succinate; polyvinyl acetate phthalate (PVAP); acrylic based polymers; sodium alginate; polyvinyl acetate phthalate (e.g. those commercially available as e.g. the Opadry® range); shellac; fatty acids; waxes; plant fibers; carbopol; arabic gum; agar; guar gum; locust bean gum; carrageenan; medium chain fatty acids; locust beans; and xanthan gum. One or more of these materials may be used to prepare a suitable enteric coating.

Suitably, the formulation is capable of releasing the FSVs at an appropriate pH, for example at pH 5.5-9 (in the intestines) instead of at pH 1-5 (in the stomach). Such release properties can be provided by an enteric coating, for example.

### EXAMPLES

An exemplary formulation is set out in the below table. The amounts shown are per 0.25 mL dose.

| Ingredient | Amount in mg (IU) | |
|---|---|---|
| Vitamin A (retinyl palmitate) | 1.5 | (5000) |
| Vitamin D3 (cholecalciferol) | 0.0375 | (1500) |
| Vitamin E (DL alpha tocopheryl acetate) | 100 | (150) |

| | |
|---|---|
| Vitamin K1 (phytomenadione) | 5 |
| Medium chain triglyceride | Remainder |

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

Unless stated otherwise, the application contemplates preparation, storage and use of the present formulations at room temperature (e.g. below 25 C) and atmospheric pressure.

Any of the preferences or aspects described herein can be combined with any other preference or aspect unless context demands otherwise.

### REFERENCES

A number of publications are cited above in order to more fully describe and disclose the invention and the state of the art to which the invention pertains. Full citations for these references are provided below. The entirety of each of these references is incorporated herein.

Nutritional Management of Cystic Fibrosis, 2^{nd} Edition—September 2016 from the Cystic Fibrosis Trust.

Encyclopedia of Dietary Supplements, 2^{nd} Edition, Coates et al. (2010), pages 778-860.

1. A capsule, tablet or pill comprising a formulation comprising:
at least two of the fat-soluble vitamins vitamin A, vitamin D, vitamin E and vitamin K; and
one or more kinds of medium-chain triglyceride.

2. A capsule, tablet or pill according to claim 1, wherein: vitamin A is present as active in an amount of between 500 and 2500 mcg per capsule, tablet or pill; and/or vitamin D is present as active in an amount of between 10 and 75 mcg per capsule, tablet or pill; and/or vitamin E is present as active in an amount of between 20 and 200 mg per capsule, tablet or pill; and/or vitamin K is present as active in an amount of between 1 and 10 mg per capsule, tablet or pill.

3. A capsule, tablet or pill according to claim 1, wherein vitamin A comprises retinyl palmitate; and/or vitamin D comprises cholecalciferol; and/or vitamin E comprises DL-alpha tocopheryl acetate and DL-alpha tocopherol; and/or vitamin K comprises phylloquinone.

4. A capsule, tablet or pill according to claim 1, comprising each of the fat-soluble vitamins.

5. A capsule, tablet or pill according to claim 1, which has an enteric coating.

6. A capsule according to claim 1.

7. A food or drink comprising the capsule, tablet or pill of claim 1.

8. (canceled)

9. A pharmaceutical composition comprising the capsule, tablet or pill according to claim 1 and a pharmaceutically acceptable diluent or carrier or excipient.

10. (canceled)

11. A method of treating a patient comprising administering an effective amount of the capsule, tablet or pill according to claim 1 to the patient.

12. A method of treating a patient suffering from cystic fibrosis comprising administering an effective amount of the capsule, tablet or pill according to claim 1 to the patient.

13. A method of treating a patient comprising administering an effective amount of the food or drink of claim 7 to the patient.

14. A method of treating a patient suffering from cystic fibrosis comprising administering an effective amount of the food or drink of claim 7 to the patient.
